(19) United States
(12) Patent Application Publication
(10) Pub. No.: US 2016/0000504 A1
(43) Pub. Date: Jan. 7, 2016

(54) METHOD FOR TRAINING IMMUNE CELLS USING MILLIMETER WAVE THERAPY

(71) Applicant: Electrohealing Holdings, Inc., Estero, FL (US)

(72) Inventors: Michael R. Manning, Langhome, PA (US); Michael Weiner, Estero, FL (US)

(21) Appl. No.: 14/658,150

(22) Filed: Mar. 14, 2015

Related U.S. Application Data
(60) Provisional application No. 61/953,371, filed on Mar. 14, 2014.

(51) Int. Cl. A61B 18/18; A61N 5/00

(52) U.S. Cl. CPC A61B 18/18; A61N 5/00; A61B 2018/1807; A61B 2018/00791

(57) ABSTRACT

A method is provided that trains the immune system to resist cancer. A tumor of a biological organism is irradiated with millimeter waves having a prescribed frequency and power density. The biological organism thereafter exhibits an increased level of resistance to the growth of additional tumors, even at remote locations in the body.

### CROSS REFERENCE TO RELATED APPLICATIONS

This application claims priority to the U.S. Provisional Application No. 61/953,371, filed Mar. 14, 2014, entitled METHOD FOR TRAINING IMMUNE CELLS USING MILLIMETER WAVE THERAPY, the contents of which are relied upon and incorporated herein.

### BACKGROUND OF THE INVENTION

The subject matter disclosed herein relates to a method for training immune cells using millimeter wave therapy. Millimeter wave therapy is a treatment that triggers a series of biological effects in response to local skin exposure to millimeter-wavelength electromagnetic waves. Various forms of the therapy have been used in Eastern European countries for several decades but the therapy has not been widely adopted by Western countries. “Physiological Mechanism Underlying Millimeter Wave Therapy” in: Ayrapetyan, S. N. et al. Bioelectromagnetics (Springer, 2006), pp. 241-251.

Cancer immunotherapy is a treatment regimen that triggers a biological organism's immune response to treat cancer. For example, Bacillus Calmette-Guerin (BCG) may be introduced into the bladder of an individual suffering from bladder cancer. The individual's immune response to the BCG not only treats the bacterial infection but also attacks the cancer cells lining the walls of the bladder. Unfortunately, the immune response is localized (e.g. to the bladder) and is often specific to a particular strain of cancer. Cancer immunotherapy generally relies on the presence of cell surface receptors that are present on a surface of cancer cells but are infrequent (or absent) on healthy cells. Provoking an appropriate immune response has therefore been a difficult objective to accomplish.

The discussion above is merely provided for general background information and is not intended to be used as an aid in determining the scope of the claimed subject matter.

### SUMMARY OF THE INVENTION

Accordingly, the present invention provides methods and apparatus for inducing an immune response to the presence to cancerous cells in an organism, such as a mammal. In some embodiments, a method is provided that trains the immune system to resist cancer. A tumor of a biological organism, such as the body of a mammal, is irradiated with millimeter waves having a prescribed frequency and power density. The biological organism thereafter exhibits increased resistance to the growth of additional tumors, even at remote locations in the body. The increased resistance is observed against tumors of a strain unlike the strain of the original tumor. One advantage of the disclosed method is that millimeter waves are non-ionizing and are therefore healthier for a patient. Millimeter waves do not cause the unhealthy effects often caused by radiation exposure (e.g. cancer radiation therapy, etc.).

According to some embodiments of the present invention, millimeter wave therapy may be applied to a living organism, including a mammal, to kill cancer cells and tumors without altering the chemical makeup of the cancer cells, but causing them to fragment into small pieces, such that the organism's immune system is effective against the cancer cells.

In another aspect, this disclosure provides therapeutic methods for breaking up glycoproteins of the tumor wall, so that an organism's immune system learns to attack the tumor cells.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A, FIG. 1B and FIG. 1C are graphs showing change in tumor size for certain groups of mice;

FIG. 2A and FIG. 2B are graphs showing the inhibitor effect of an opioid antagonist on MMWT on mice;

FIG. 3 is a schematic depiction of a system for performing a first stage of treatment; and

FIG. 4 is a schematic depiction of a system for performing a second stage of treatment.

### DETAILED DESCRIPTION OF THE INVENTION

The idea of cancer occurring as an accumulation of mutations in a single cell goes back many decades. Researchers who have assimilated a growing mass of laboratory results and theoretical insights into six characteristics that a cancer cell must acquire in order to develop into the structure known as a malignancy or cancer tumor. The cancer cell must acquire the ability to stimulate its own growth and multiplication independently of restraints in its own environment. The cancer has to perform the process of uncontrolled or abnormally controlled growth by ignoring metabolic signals ordering it to cease mitotic activity (this is where the messages created by oncogenes and other tumor suppressors are not responded to). The cancer cell must learn to circumvent the fail-safe mechanisms that cause even slightly disabled cells to destroy themselves. It must also defeat its own internal mitotic counters, the telomeres found on the ends of chromosomes which by shortening during each cell mitosis count and then limit the number of the times a normal, non-cancer cell is allowed to divide. Therefore, in effect, without this limitation, the cancer cell becomes immortal. Also, the cancer cell must learn to initiate angiogenesis, the creation of its own network of food and oxygen providing blood vessels in the developing tumor. Finally, it must learn how to consume by dissolving surrounding tissue to further feed the growing tumor.

The cancer cells of the metastatic tumor commonly also modify the structure of their outer membrane in order to prevent the immune system from recognizing them as foreign bodies and attacking them. They exhibit a unique structure of glycoproteins on the outer surfaces of their outer membranes whose structures are supported by the metabolic activities of the living cancer tumor cells. If they happen to die or be killed, this energetic support structure is destroyed and the glycoproteins on the outer membrane rearrange themselves into a disorganized state which is then recognizable by the immune system as a foreign body.

Without wishing to be bound to any particular theory, the disclosed method is believed to modify the structure of the outer cell surfaces of the cancer tumor cells into a recognizable foreign body state by slowly heating these tumor cells to about 47° C. with millimeter waves. This causes the tumor cells to undergo a slow death and structural decomposition process known as apoptosis. During this process, the cancer cell's nucleus receives a signal which causes it to disassemble in a way which allows the disorganized glycoproteins on the cell's outer membrane to remain structurally intact. The cancer cell membrane fragments are then released into the lymphatic systems structurally intact.

The teacher or dendritic cells of the immune system acquire glycoprotein coated cell fragments and transfer the relevant markers unique to cancer cells to their own outer membranes. They, in turn contact T, Band NK (natural killer cells) of the immune system and, by transferring the relevant cancer identifier glycoproteins to these cells, instruct them how to recognize and attack other metastatic cells at any location in the body where tumors have grown. From our laboratory studies, it appears that a wide range of marker types are transferred to these immunocytes and so many different tumor cell types are then susceptible to destruction by the activated T, B, and NK cells of the immune system.

Millimeter wave therapy (MMWT) has been shown to inhibit melanoma in mice. See Radzievsky et al. Millimeter Wave-Induced Suppression of B16 F10 Melanoma Growth in Mice: Involvement of Endogenous Opioids, 2004, Bioelectromagnetics 25:466-473. Mice were separated into eight groups with at least ten mice per group. The MMWT groups were injected with B16 melanoma cells and, after a predetermined delay, subsequently irradiated with millimeter wave therapy (61.22 GHz, power density of 13.3×10-³ W per square cm using a G4-142 “Istok” wave generator) for fifteen minutes. The irradiation step was repeated once a day or five times a day, as shown in Table 1, for the following four days.

TABLE 1
Inhibition of tumor growth using millimeter wave therapy

| | | # of | Delay before | |
|---|---|---|---|---|
| Group | MMWT? | irradiations per | first | Comment |
| 1 | Yes | 1 | 30 min | |
| 2 | No | 0 | n/a | Control for group 1 |
| 3 | Yes | 5 | 5 days | |
| 4 | No | 0 | n/a | Control for group 3 |
| 5 | Yes | 5 | 10 days | |
| 6 | No | 0 | n/a | Control for group 5 |
| 7 | Yes | 5 | 5 days | Naloxone HCl |
| 8 | No | 0 | 5 days | Naloxone HC; |
| | | | | Control for group 7 |

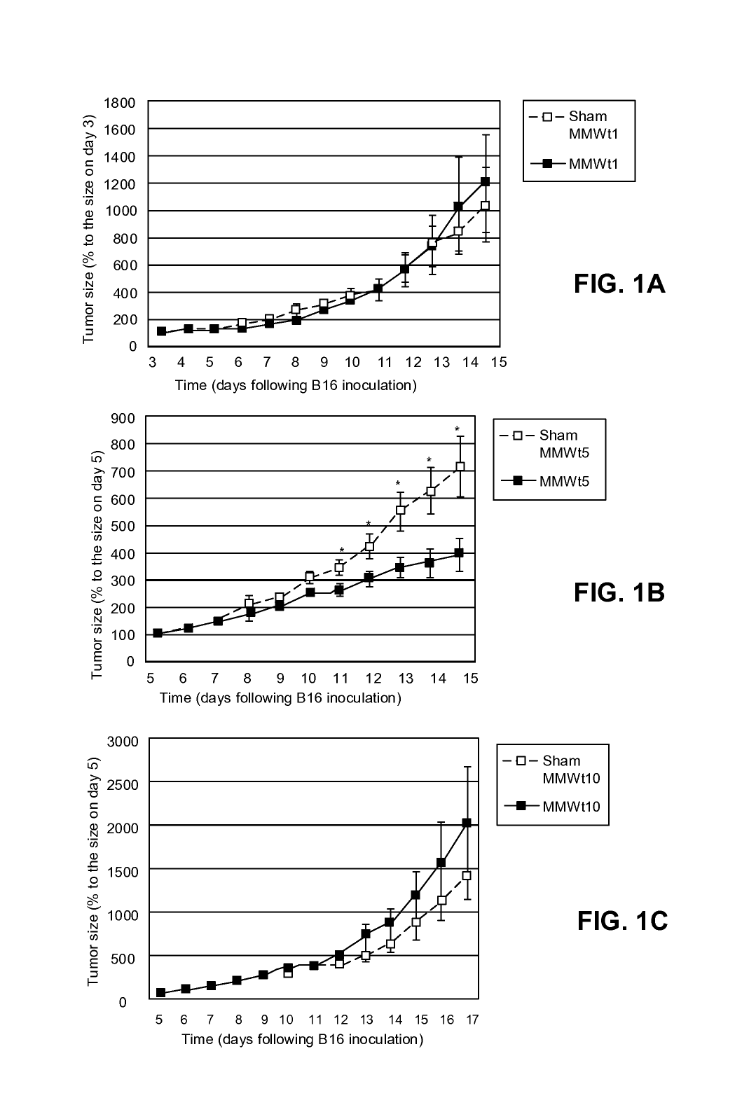

Two-dimensional tumor size was monitored over time for each group. FIG. 1A is a graph showing change in tumor size over time for groups one and two (thirty minute delay before MMWT). FIG. 1B is a graph showing change in tumor size for groups three and four (5 day delay before MMWT). FIG. 1C is a graph showing change in tumor size for groups five and six (10 day delay before MMWT). Group one (thirty minute delay) showed no change in tumor size relative to the control group two. Groups five and six (10 day delay) also showed no statistical difference in tumor size. Groups three and four (5 day delay), however, showed a statistically meaningful inhibition in tumor growth due to the MMWT. The two-dimensional area of the MMWT-treated tumors ranged from 42.7-667% the size of the non-MMWT-treated tumors. When the three-dimensional size of the tumors was approximated using tumor weight, over a threefold reduction in tumor size was found.

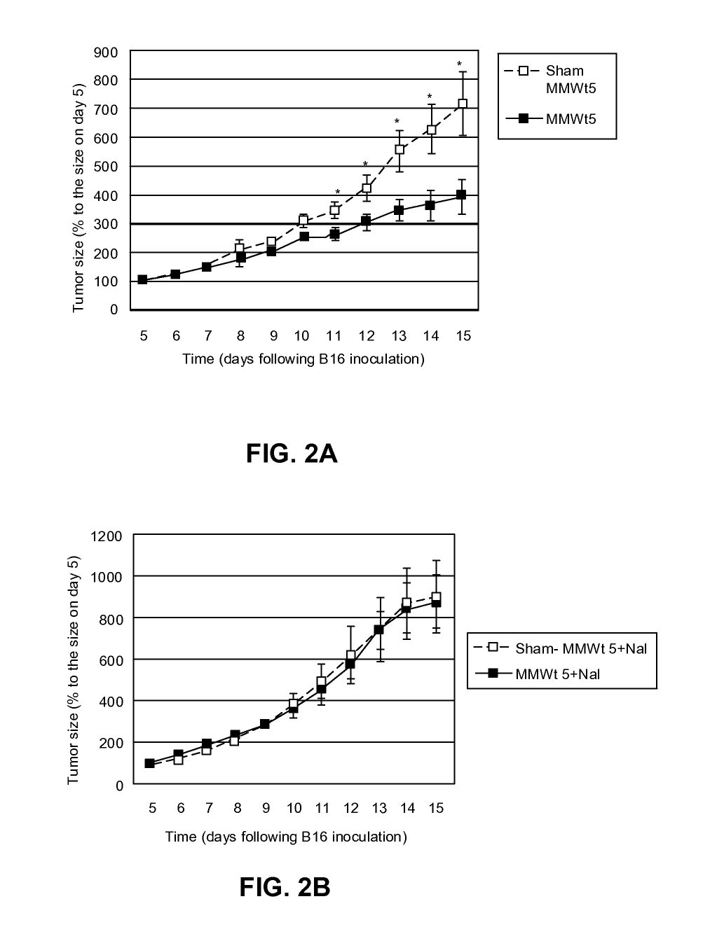

Without wishing to be bound to any particular theory, the inhibition in tumor growth may be caused by an immune response that is initiated by the millimeter wave therapy. Since opioids are known to be modulators of immune responses, groups seven and eight were given 1 mg per kg of an opioid antagonist, naloxone hydrochloride, thirty minutes prior to each treatment. These groups were otherwise treated in a manner substantially identical to groups three and four. FIG. 2A is a graph showing change in tumor size for groups three and four (5 day delay before MMWT). FIG. 2B is a graph showing change in tumor size for groups three and four (5 day delay before MMWT, treating with naloxone). Comparison of FIG. 2A and FIG. 2B demonstrates the opioid antagonist blocks the beneficial effects of MMWT. The data suggests MMWT of a melanoma tumor provokes an immune response that inhibits tumor growth. In one embodiment, the biological organism is substantially free of opioid antagonists (i.e. not taking additional medications that inhibit opioids beyond the normal level of inhibition when not taking such medications).

Disclosed herein is a method for training immune cells of a biological organism to inhibit cancer. After irradiation of a first tumor using millimeter wave therapy, second tumors elsewhere in the body may also be inhibited due to the presence of the trained immune cells, including those tumors which are from different strains.

Without wishing to be bound to any particular theory, chemical surface receptors are believed to be exposed to the biological organism's immune system and immune cells (e.g. T-cells, antibodies) then become trained to recognize the chemical surface receptors. The method is non-radiative and avoids excessively heating cells and destroying or altering their chemical surface receptors. Traditionally, therapeutic frequencies used in MMWT are 61.22 GHz, 53.57 GHz and 42.25 GHz. Notwithstanding this convention approach, frequencies of about 35 GHz±3 GHz have proven to be substantially more effective for the disclosed method than the prior art's frequencies. Without being bound by a particular theory, it is possible the 35 GHz frequency is more effective as water minimally absorbs millimeter waves at this frequency leading to a greater depth of penetration in tissue. It is noteworthy there is also relatively low millimeter wave absorption by water at about 94 GHz.

In a second experiment malignant melanoma tumors were implanted into the skin of balb/c mice while simultaneously injecting malignant mesothelioma cancer cells into the tail vein. The injected malignant mesothelioma cancer cells were selected to preferentially lodge in the lungs of these mice. If left untreated, 100% of the mice would die of a loss of lung function in 20-25 days. If the implanted tumor was treated with millimeter wave therapy (35 GHz, power density of 1-10 W per square cm, one application for thirty minutes), a portion of the implanted tumor would undergo apoptosis over a one to two-day period and release chemical surface receptors. The mice's immune system was then able to recognize and attack both the implanted cancer cell as well as the injected cancer cells in the lungs, despite the millimeter waves being unable to penetrate to such deep tissue. Lung cancer tumors never developed in the mice that were subjected to this MMWT.

In a third experiment, the effects of millimeter wave therapy on ionic charge density on the surfaces of living algae cells was determined. A four-pole rotating electrodynamic field was created by successively driving four small platinum electrodes (positioned at ninety degree angles) with sequential positive pulses. Cells of erithrospera algae were placed in water at the center of this rotating electrodynamic field. A broad resonant rotating of the algae was observed when the field was rotated at speeds of 0.75-1.5 MHz. In response to this applied field, the algae would rotated at speeds of 5-10 rotations per minute. MMWT-treated algae (35 GHz, power density of 10-² W per square cm for thirty minutes) showed at 20-50% reduction in rotation speed, suggesting a reconfiguration or removal of chemical surface receptors as a result of MMWT.

In one embodiment, the method trains immune cells such as T cells, B cells and/or natural killer (NK) cells. Without wishing to be bound to a particular theory, T cell and B cells are believed to be the likely cause of the observations. The effects on the injected cancer cells in the lungs of the mice is unlikely to be the direct result of MMWT, as millimeter waves do not generally penetrate to such depths. Accordingly, changes in the chemical surface receptors on the lung tumors and subsequent attack by NK cells is unlikely to be the mechanism of action. Training of T cells and or B cells to recognize certain chemical surface receptors in the implanted cancer cells (released or presented due to the MMWT treatment) and subsequent migration of those trained cells to the injected lung tumors is consistent with the observations.

In one embodiment, the first tumor is a skin tumor and the irradiation occurs on an external surface of the skin with millimeter waves of a specific frequency, power and application conditions. In another embodiment, an endoscope, a catheter or laparoscopic surgery is used to deliver the millimeter waves at a location other than the external surface of the skin. In one embodiment, the millimeter waves are delivered by a handheld emitter that includes an optical sensor, a temperature sensor and a processor operatively connected thereto. The optical sensor locates an area of dark skin (e.g. a tumor) and supplies millimeter waves. The temperature sensor monitors the delivery of the millimeter waves to provide proper temperature control.

In another embodiment, a tumor is simultaneously treated with MMWT while subjected to mechanical stress that further weakens cellular membranes within the tumors. This further promotes release of chemical surface receptors and enhances the immune system training. Generally, cancer cells are distended compared to normal cells of the same type and have thinner cell membranes that are more susceptible to disruption when subjected to mechanical stress. For example, ultrasound may be delivered from a lithotripter during the MMWT to preferentially disrupt the cancer cell membrane without disturbing the integrity of the normal, non-malignant adjacent cells. See U.S. Patent Publication No. 2012/0225475 entitled “Cytometry System with Quantum Cascade Laser Source, Acoustic Detector, and Micro-Fluidic Cell Handling System Configured for Inspection of Individual Cells.”

In another embodiment, the immune system's response to tumor cells is promoted using the simultaneous delivery of MMWT and infrared wavelengths (0.0007 mm to 1 mm) to stimulate the production of chemical surface receptors for teaching the immune-responsive cells to attack cancer cells Infrared energy may be used to release chemical surface receptors through the process of apoptosis without overheating the tumor cells to the point where the organic components of the cell's membrane fragments are destroyed or altered in structure. These chemical surface receptors then circulate through the lymphatic circulatory systems of the biological organism and are recognized by the immune system.

In the disclosed method, the millimeter waves are believed to have two effects: first, the thermal initiation of the apoptotic reaction in melanoma cells released cell fragments into the lymphatic system and second, lower level irradiation in the penumbra of the millimeter wave beam activated lymphocytes in the biological organism's immune system. These activated lymphocytes then received protein structures from the apoptotically released tumor cell fragments and presented them to lymphocytes. These lymphocytes, in turn, attacked cancer cells at remote locations in the animal's body. In one embodiment, the method is preformed while the biological organism is not being treated with a chemotherapeutic agent, as such treatments can suppress the organism's immune response. The millimeter waves have a frequency of 35 GHz±3 GHz and have a sinusoidal waveform. The power density is generally between 10-² W per square centimeter and 10 W per square centimeter.

In one embodiment, a probe with a millimeter wave source is inserted into a biological organism to treat a tumor located within the body. Such an embodiment is useful for tumors that are located within the body, as millimeter waves are generally restricted to a tissue depth of about one millimeter. For example, laparoscopy may be used to perform MMWT on an internal organ, such as prostate, breast, lung and pancreatic cancer.

In another embodiment, millimeter waves are used to treat melanoma cancer cells on the surface of the skin. Since tissue depth penetration of MMWT is limited to about one millimeter, non-cancerous tissue located beneath the cancer cells on the skin surface are not affected.

In one embodiment, the method comprises a two-stage treatment. In a first stage, an open end waveguide is used to direct millimeter waves directly to a tumor as described elsewhere in this specification. The power density may be sufficient to increase the temperature of the treated area by about 5-10° C. to promote apoptosis without altering the chemical structure of cell fragments. In one embodiment, the temperature is increased to about 47° C. at the treated area for about thirty minutes. In a second stage, a horn is used to provide millimeter waves to the entire biological organism to stimulate the organism's lymphatic system. During the second stage, the power density of the MMWT treatment is less than the first stage. For example, the treatment during the second stage may be performed at 35 GHz at a power density of 10-² W per square centimeter for 15 minutes for twenty days, thirty minutes per day. The power density during the first stage may be, for example, 1-10 W per square centimeter. To ensure whole-organism treatment, the millimeter wave source may be placed at a distal location relative to the organism (e.g. 1-2 meters away).

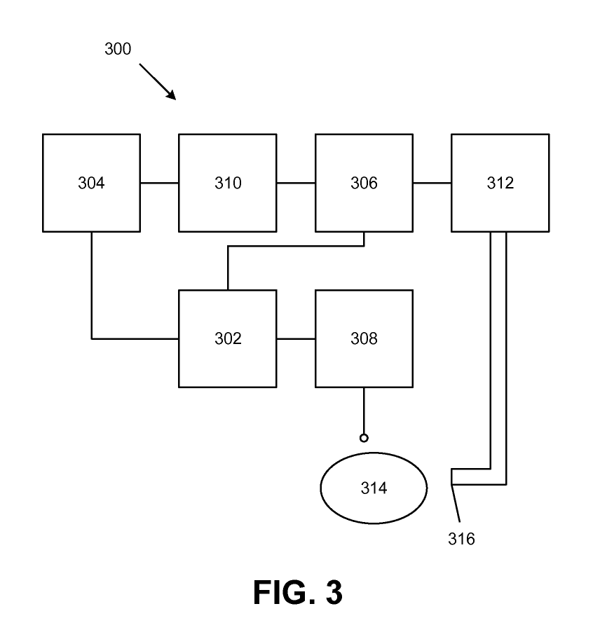

FIG. 3 is a schematic diagram of a system 300 for performing the first stage of treatment. A programmable controller 302 operates a timer 304, an attenuator 306 and receives temperature data from a temperature sensor 308 operatively connected to a subject 314. The temperature sensor 308 may be, for example, an IR thermometer/transmitted from Omega Engineering, Inc. of Stanford, Conn. (part number OS551A-MA-6). The attenuator 306 attenuates the power density of the millimeter waves produced by millimeter wave generator 310. The attenuator 306 may be, for example, a fixed attenuator from MILLITECH® of Northampton, Mass. (part number FXA-28-S30PN). A millimeter wave isolator 312 (e.g. a filter) selects for a desired frequency of millimeter waves. The attenuated and selected millimeter waves are then delivered to subject 314 by a focusing aperture 316 in a timing sequence controlled by timer 304. The focusing aperture is configured to delivery millimeter waves to a specific area of the body of the subject 314. A straight waveguide from MILLITECH® may be used.

In one embodiment, the focusing aperture 316 is disposed on a probe that may be inserted into the biological organism. The probe may be inserted by, for example, laparoscopy, to provide millimeter wave treatment to a variety of internal organs including the colon, the prostate or soft tissue such as breast tissue. When additional room is desired within the abdominal cavity, air may be introduced to inflate the abdominal cavity.

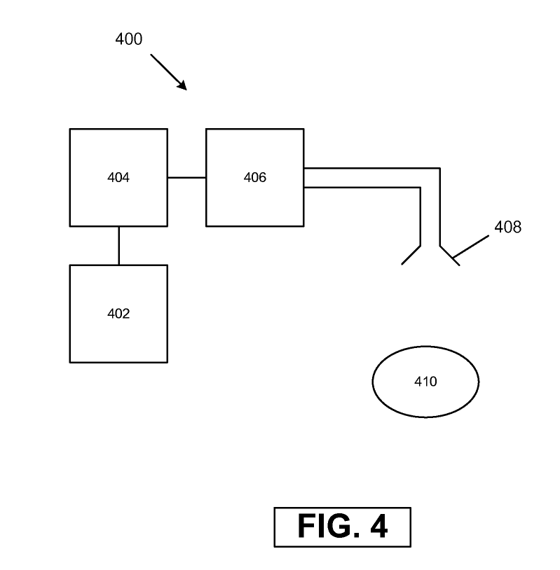

FIG. 4 is a schematic diagram of a system 400 for performing the second stage of treatment. A programmable controller 402 operates a timer 404 that controls a millimeter wave generator 406. Millimeter waves produced by the millimeter wave generator 406 are directed to the subject 410 by horn 408. The horn 408 is configured to deliver millimeter waves to the entire body of the subject 410. The horn may be, for example, a standard gain horn from MILLITECH® of Northampton, Mass. (item no. SGH-28-SPOOO). In one embodiment, the system 400 further comprises an attenuator and a millimeter wave isolator to control the power density and frequency of the millimeter waves.

In one embodiment, the immune cells from a first individual are trained to recognize tumor cells. These trained immune cells are then transferred to a second individual who may or may not have the same cancer condition as the first individual. Exposure of the second individual to the trained immune cells provides the second individual with an increased resistance to tumor cells.

The use of disclosed method to treat other skin disorders is also contemplated. For example, the method may be used to treat warts, including genital warts, scars, calluses, and the like.

Having defined the fundamental method of treatment using nonionizing radiation to then induce immunization, we now turn our attention to various methods of administration and application.

The first is a handheld device which will allow the operator to administer the millimeter wave radiation directly to a lesion or a tumor. The handheld device can include controls for varying the frequency and intensity of the energy emission, the width of the beam for treating a large precancerous tumor, then a small precancerous tumor, a sensor for identifying and controlling the heat of the energy induced by the treatment, the ability to provide additional treatment such as ultraviolet in conjunction with millimeter waves, and if desired the ability to also emit an ionizing output to ensure destruction of resistant tumors.

An enhancement to this device includes the ability to sense cancerous cells using any of a variety of means including multi-photon optics, phase-based microscopy, and other means which allowed the identification of cells and their analysis to replicate the function or as close as possible as human radiologists can provide.

When a cell is identified as cancerous, the treatment will be given to that area. Certain systems can map the area of cancer cells using a variety of means such as is found in MRI, CAT scan, PET scan, and other systems which construct the XYZ coordinates of a person or animal receiving treatment. This allows replication on a daily basis of the treatment.

Cancer cells that are not on the skin can be reached by a variety of means including and the scopes and catheters which carry the emitter to the point of need, and have the necessary power to generate the output. This can also use waveguides to examine beneath the skin to the point where a tumor or cancerous cell cluster exists.

A means of providing treatment which would not be subject to the electromagnetic fields found in devices such as MRI can be found in U.S. Pat. Nos. 6,725,092; 6,846,985; 6,980,848 and 7,729,777, hereby incorporated by reference.

A means to treat blood and lymph fluids which may contain cancerous cells such as leukemia, or metastasizing cancer cells from any source can be treated by providing this treatment to the blood itself while flowing into the vessel. This can, for example, be radiation (using non-ionizing radiation) to the vessel such as a capillary or veins or arteries and the rest, the carotid artery, the ankle, and other places where close exposure to flowing blood is possible. The length of treatment of the blood may be longer than the time to directly treat a tumor since the cells are constantly circulating. However, because non-ionizing radiation is used, no negative results are anticipated.

There has been much success over time in the use of ultraviolet waves to treat disease including some cancers. In one embodiment, the device further includes an ultraviolet emitter or another frequency which shows efficacy in combination with the disclosed method.

There is much indication of the potential success of ultrasound in treating cancer. In one embodiment ultrasound in, combination with the disclosed methodology, can improve the overall efficacy. This may be due to some ultrasound frequencies causing a weakening in the mitotic spindles of the cell. Additionally energy fields such as millimeter waves can be modified by ultrasound, and the reverse—an ultrasound—focused energy beam can be modulated at millimeter wave frequencies so that the energy beam can penetrate deeply due to the influence of one set of waves modulating the other.

Additionally various means of response and observation to determine the position of and treatment of cancer cells in tissue can be used to ensure that the treatment covers the boundaries as well as the core of a cancerous growth.

The disclosed method is fully MRI compatible and does not interfere with imaging such that the patient can receive millimeter wave treatment while under MRI observation. It is also possible for powerful device such as an MRI signal to be captured and reread at a different frequency. For example, the 64 MHz frequency of an MRI can be increased to the desired 32 to 48 GHz range.

Each of these delivery devices for providing the cancer/tumor growth reversal without the negative effects of radiation and chemotherapy can also be applied by automatic devices such as the Davinci surgical robot by INTUITIVE SURGICAL®, or other devices designed to deliver, position, treat, track and apply therapeutics to the patient. The advantage of a robotic device such as the Davinci is a much greater degree of position accuracy and to overcome the tremors that can cause unintended position anomalies. The Davinci is known for 10 mm accuracy where humans tend closer to 100 mm, a delta than can mean a lot in terms of risk for a patient undergoing a prostate procedure or brain surgery. Position accuracy and detection and treatment combinations that allow cancer tissue at the site to be treated based on real time analysis near the probe is possible.

Similarly a millimeter wave device delivered by endoscopy or catheter, or MRI or CatScan guided surgery, can detect and treat target cells (cancer, et al), in real time at the site. Repeat treatments can be directed to the previously detected site and tissue.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for training immune cells of a biological organism, the method comprising:
irradiating a tumor of a biological organism with a first dose of millimeter waves having a first frequency between 30 and 48 GHz at a power density of about 1 to about 10 W per square centimeter for between 1 min and 60 min;
irradiating the biological organism with a second dose of millimeter waves having a second frequency between 30 and 48 GHz at a power density of about 10-² to about 1 W per square centimeter for between 1 min and 60 min;
permitting at least one day to pass to permit at least some cells within the tumor to undergo apoptosis and release cellular contents;
letting the cellular contents become exposed, in vivo, to immune cells of the biological organism after the second dose of millimeter waves has been irradiated; and
allowing the immune cells to become trained to recognize the cellular contents of the tumor.

2. The method as recited in claim 1, wherein the tumor is a melanoma tumor.

3. The method as recited in claim 1, wherein the first frequency is between 32 and 38 GHz.

4. The method as recited in claim 1, wherein the second frequency is between 32 and 38 GHz.

5. A method for training immune cells of a biological organism, the method comprising:
inserting a probe into a biological organism, wherein the probe has a source for emitting millimeter waves;
irradiating a tumor on a tissue disposed within the biological organism with millimeter waves from the source, wherein the millimeter waves have a frequency between 30 and 48 GHz at a power density of about 10-² W per square centimeter for between 1 min and 60 min;
permitting at least one day to pass to permit at least some cells within the tumor to undergo apoptosis and release cellular contents;
letting the cellular contents become exposed, in vivo, to immune cells of the biological organism; and
allowing the immune cells to become trained to recognize the cellular contents of the tumor.

6. The method as recited in claim 5, wherein the step of irradiating is repeated at least once a day for at least three days.

7. The method as recited in claim 5, wherein the tumor is a colorectal tumor.

8. The method as recited in claim 5, wherein the tumor is a prostate tumor.

9. The method as recited in claim 5, wherein the tumor is a breast tumor.

10. The method as recited in claim 5, wherein the step of inserting the probe further comprises a step of inflating an abdominal cavity of the biological organism with air.

11. The method as recited in claim 5, wherein the biological organism is a human being.

12. A method for training immune cells of a human the method comprising:
irradiating a melanoma tumor on a skin surface of a human with millimeter waves having a first frequency between 30 and 48 GHz at a power density of about 10⁻² to about 10 W per square centimeter for between 1 min and 60 min;
permitting at least one day to pass to permit at least some cells within the melanoma tumor to undergo apoptosis and release cellular contents;
letting the cellular contents become exposed, in vivo, to immune cells of the human;
allowing the immune cells to become trained to recognize the cellular contents of the melanoma tumor.

13. The method as recited in claim 12, wherein the power density is between about 1 to about 10 W per square centimeter.

14. The method as recited in claim 12, further comprising sequential steps of:
waiting for at least one day after the step of letting;
exposing the human to additional millimeter waves having a second frequency between 30 and 48 GHz at a power density of about 10-² W per square centimeter for between 1 min and 60 min;
repeating the step of exposing at least once a day for at least ten days.

15. The method as recited in claim 14, wherein the human exhibits increased resistance to non-melanoma tumors relative to a human who has not experienced the step of irradiating.

16. The method as recited in claim 14, wherein the frequency is about 35 GHz.

17. The method as recited in claim 14, wherein the step of repeating is performed at least once a day for at least twenty days.

18. The method as recited in claim 14, wherein the human is substantially free of opioid antagonists during the method.

19. An apparatus for delivering millimeter waves to a biological organism for training immune cells of the biological organism, the apparatus comprising:
a millimeter wave generator for producing millimeter waves; and
a programmable controller, operatively connected to: a timer that controls the timing of the millimeter waves, an attenuator that controls the power density of the millimeter waves; and
a millimeter wave isolator that controls the frequency of the millimeter waves;
the apparatus providing the millimeter waves having a frequency between 30 and 48 GHz at a power density of about 10⁻² to about 10 W per square centimeter for between 1 min and 60 min.

20. The apparatus as recited in claim 19, further comprising a horn for spreading the millimeter waves over an area that encompasses the biological organism's whole body.
